(19) United States
(12) Patent Application Publication
(10) Pub. No.: US 2013/0006134 A1
(43) Pub. Date: Jan. 3, 2013

(54) METHODS AND SYSTEMS FOR MONITORING VOLUMETRIC CARBON DIOXIDE

(75) Inventors: Peter R. Doyle, Vista, CA (US); Gardner Kimm, Carlsbad, CA (US); Mehdi M. Jafari, Laguna Hills, CA (US)

(73) Assignee: Nellcor Puritan Bennett LLC, Boulder, CO (US)

(21) Appl. No.: 13/174,240

(22) Filed: Jun. 30, 2011

(51) Int. Cl. A61B 5/08

(52) U.S. Cl. 600/532

(57) ABSTRACT

This disclosure describes novel systems and methods for monitoring volumetric CO₂ during ventilation of a patient being ventilated by a medical ventilator. The disclosure describes more accurate, more cost effective, and/or less burdensome non-invasive methods and systems for calculating volumetric CO₂ than previously utilized methods and systems. The disclosure describes estimating a flow rate in a breathing circuit to calculate a volumetric CO₂. Further, the disclosure describes synchronizing the estimated flow rate with a measured CO₂ to calculate a volumetric CO₂. Additionally, the disclosure describes synchronizing a measured flow rate from within the breathing circuit with a measured CO₂ to calculate a volumetric CO₂.

Medical ventilators may determine when a patient takes a breath in order to synchronize the operation of the ventilator with the natural breathing of the patient. In some instances, detection of the onset of inhalation and/or exhalation may be used to trigger one or more actions on the part of the ventilator. Accurate and timely measurement of patient airway pressure and lung flow in medical ventilators are directly related to maintaining patient-ventilator synchrony and spirometry calculations and pressure-flow-volume visualizations for clinical decision making.

In order to detect the onset of inhalation and/or exhalation, and/or obtain a more accurate measurement of inspiratory and expiratory flow/volume, a flow or pressure sensor may be located close to the patient. For example, to achieve timely non-invasive signal measurements, a differential-pressure flow sensor may be placed at the patient wye proximal to the patient. However, the ventilator circuit and particularly the patient wye is a challenging environment to make continuously accurate measurements.

Other sensors for monitoring the patient and ventilation may be located in the patient circuit. In some systems, carbon dioxide (CO₂) levels in the breathing gas from the patient are measured. Many of these previously known medical ventilators display the monitored CO₂ levels of the breathing gas from the patient.

### Monitoring Volumetric Carbon Dioxide

This disclosure describes novel systems and methods for monitoring volumetric CO₂ during ventilation of a patient being ventilated by a medical ventilator. The disclosure describes more accurate, more cost effective, and/or less burdensome non-invasive methods and systems for calculating volumetric CO₂ than previously utilized methods and systems. The disclosure describes estimating a flow rate in a breathing circuit to calculate a volumetric CO₂. Further, the disclosure describes synchronizing the estimated flow rate with a measured CO₂ to calculate a volumetric CO₂. Additionally, the disclosure describes synchronizing a measured flow rate from within the breathing circuit with a measured CO₂ to calculate a volumetric CO₂.

In part, this disclosure describes a method for monitoring volumetric CO₂ during ventilation of a patient being ventilated by a medical ventilator. The method includes:

a) estimating at least one flow rate at a first location in a breathing circuit by monitoring at least one respiratory parameter with at least one sensor located outside of the breathing circuit;

b) monitoring CO₂ concentrations with a capnometer at a second location in the breathing circuit; and

c) calculating a volumetric CO₂ passing through at least one of the first and second locations for at least one breath based at least on an algorithm, the monitored CO₂ concentrations taken by the capnometer, and the at least one estimated flow rate.

Yet another aspect of this disclosure describes a medical ventilator system including: a pneumatic gas delivery system; a sensor estimator; a capnometer; and a processor. The pneumatic gas delivery system is adapted to control a flow of gas from a gas supply to a patient via a breathing circuit. The sensor estimator estimates at least one flow rate at a first location in the breathing circuit based at least on measurements taken by at least one sensor located outside of the breathing circuit. The capnometer monitors an amount of carbon dioxide in respiration gas at a second location in the breathing circuit. The processor is in communication with the pneumatic gas delivery system, the sensor estimator, and the capnometer. Further, the processor is configured to calculate a volumetric CO₂ passing through at least one of the first and second location for at least one breath based at least on an algorithm, the monitored CO₂ concentrations taken by the capnometer, and the at least one estimated flow rate.

The disclosure further describes a computer-readable medium having computer-executable instructions for monitoring volumetric CO₂ during ventilation of a patient being ventilated by a medical ventilator. The method includes:

a) repeatedly estimating at least one flow rate at a first location in a breathing circuit by monitoring at least one respiratory parameter with at least one sensor located outside of the breathing circuit;

b) repeatedly monitoring CO₂ concentrations with a capnometer at a second location in the breathing circuit; and

c) repeatedly calculating a volumetric CO₂ passing through at least one of the first and second locations for at least one breath based at least on an algorithm, the monitored CO₂ concentrations taken by the capnometer, and the at least one estimated flow rate.

The disclosure also describes a ventilator system including means for estimating at least one flow rate at a first location in a breathing circuit by monitoring at least one respiratory parameter with at least one sensor located outside of the breathing circuit; means for monitoring CO₂ concentrations with a capnometer at a second location in the breathing circuit; and means for calculating a volumetric CO₂ passing through at least one of the first and second locations for at least one breath based at least on an algorithm, the monitored CO₂ concentrations taken by the capnometer, and the at least one estimated flow rate.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments, systems and methods described below and are not meant to limit the scope of the invention in any manner, which scope shall be based on the claims appended hereto.

FIG. 1 illustrates an embodiment of a ventilator.

FIG. 2 illustrates an embodiment of a ventilator.

FIG. 3 illustrates an embodiment of a method for monitoring volumetric CO₂ during ventilation of a patient being ventilated by a medical ventilator.

FIG. 4 illustrates an embodiment of a method for monitoring volumetric CO₂ during ventilation of a patient being ventilated by a medical ventilator.

FIG. 5 illustrates a graph of 0 milliseconds (ms) delay between estimated lung flow and CO₂ concentration signals.

FIG. 6 illustrates a graph of 60 milliseconds (ms) delay between estimated lung flow and CO₂ concentration signals.

FIG. 7 illustrates a graph of the effect a delay between estimated lung flow and CO₂ concentration signals have on volumetric CO₂ measurement error.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to monitor the rate at which breathing gas is supplied to the patient and it may be desirable to monitor the amount of carbon dioxide (CO₂) exhaled and/or inhaled by the patient. It may also be desirable to monitor the amount of volumetric carbon dioxide (VCO₂) in the respiration gas of the patient. The volumetric CO₂ is calculated on a per breath basis by utilizing the CO₂ flow over the entire breath period (i.e., inhalation and exhalation). Calculation of VCO₂ requires the concurrent measurement of flow and a concentration of CO₂.

Some systems have flow sensors in the breathing circuit, such as at the patient wye and/or proximal to the patient. Further, some systems have one or more CO₂ sensors or CO₂ measuring devices located near the flow sensor within the breathing circuit. While the location of these sensors may be close to one another, their measurements may not be completely synchronized due to various factors, such as location, calculation delays, and transmission delays. Further, because a flow rate and a percentage of CO₂ concentration are multiplied by each other and integrated over time to calculate VCO₂, any dyssynchrony between measurements can lead to significant errors as the differences caused by the dyssynchrony will be magnified by the multiplication and phase changes, thereby decreasing the accuracy of a VCO₂ calculation. Accordingly, systems and methods for synchronizing flow measurements with CO₂ concentration measurements to increase the accuracy of VCO₂ calculations are desirable.

However, the ventilator circuit and particularly the patient wye is a challenging environment to make continuously accurate measurements. The harsh environment for the sensor is caused by condensation resulting from the passage of humidified gas through the system as well as secretions emanating from the patient. Over time, the condensate material can enter the sensor tubing and/or block its ports and subsequently jeopardize the functioning of the transducer. In addition, the risk of inter-patient cross contamination has to be addressed.

To avoid maintenance issues and costs related to the use and operation of an actual flow and/or pressure sensor with its accompanying electronic and pneumatic hardware within a breathing circuit, a sensor estimator (a virtual sensor or virtual sensor module) may be utilized to estimate parameters such as wye flow and such as flow proximal to the patient in a sensorless fashion (that is, without a sensor in the patient circuit and relying, rather, on sensors internal to the ventilator that measure pressure and/or flow into and out of the patient circuit). The values for the model parameters can be dynamically updated based on ventilator settings, internal measurement, available hardware characteristics, and/or patient's respiratory mechanics parameters extracted from ventilatory data. Accordingly, systems and methods for calculating VCO₂ without the use of a pressure and/or flow sensor in the breathing circuit are desirable.

This estimated flow may be utilized in conjunction with measured CO₂ to calculate VCO₂. However, as discussed above, because flow rate and CO₂ concentrations are multiplied by each other to calculate VCO₂, any slight changes caused by unsynchronized measurements will be magnified. In some embodiments, the flow estimates in the breathing circuit are not completely synchronized with the CO₂ concentration measurements due to various factors, such as location, calculation delays, and transmission delays affecting the accuracy of a VCO₂ calculation. Accordingly, systems and methods for synchronizing estimated flow measurements with CO₂ concentration measurements for increasing the accuracy of VCO₂ calculations are desirable. It should also be noted that dyssynchrony may occur even when the measurements are taken from the same location due to reasons such as signal processing delays or differences in sensor responsiveness.

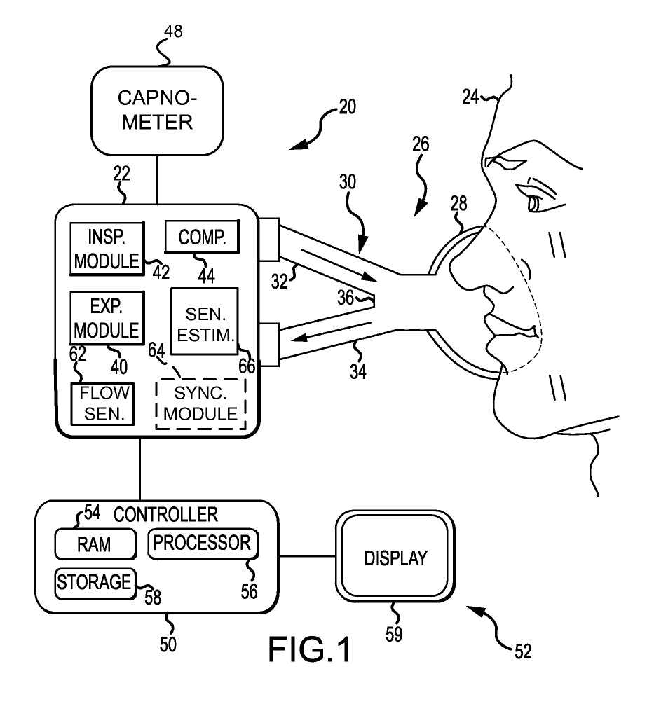

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator or breathing circuit 30. Ventilator circuit 30 could be a two-limb or one-limb circuit for carrying gas to and from the patient 24. In a two-limb embodiment as shown, a wye fitting 36 may be provided as shown to couple the patient interface 28 to the inspiratory limb 32 and the expiratory limb 34 of the breathing circuit 30.

The present description contemplates that the patient interface 28 may be invasive or non-invasive, and of any configuration suitable for communicating a flow of breathing gas from the patient circuit to an airway of the patient 24. Examples of suitable patient interface devices include a nasal mask, nasal/oral mask (which is shown in FIG. 1), nasal prong, full-face mask, tracheal tube, endotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressurized air and/or oxygen controlled through the use of one or more gas regulators) is coupled with inspiratory module 42 to provide a source of pressurized breathing gas for ventilatory support via inspiratory limb 32.

The pneumatic system 22 may include a variety of other components, including sources for pressurized air and/or oxygen, mixing modules, valves, sensors, tubing, accumulators, filters, etc. Controller 50 is operatively coupled with pneumatic system 22, signal measurement and acquisition systems, and an operator interface 52 may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

The controller 50 issues commands to pneumatic system 22 in order to control the breathing assistance provided to the patient 24 by the ventilator 20. The specific commands may be based on inputs received from patient 24, pneumatic system 22, sensors, operator interface 52 and/or other components of the ventilator 20.

In the depicted example, operator interface 52 includes a display 59. The display 59 may be touch-sensitive or voice-activated, enabling the display 59 to serve both as an input user interface and an output device. In some embodiments, the display 59 includes other input mechanisms, such as a keyboard, keypad, knob, wheel, and/or mouse. Any suitable input device for entering data by the clinician into the ventilator may be utilized by the ventilator 20.

The ventilator 20 is also illustrated as having a sensor estimator 66 (the “Sen. Estim.” in FIG. 1). The sensor estimator 66 estimates at least one respiratory parameter, such as lung flow and airway pressure, at a location in the breathing circuit 30. In some embodiments, the sensor estimator 66 estimates lung flow and/or airway pressure at the wye 36 or near the patient interface 28. In some embodiments, the sensor estimator 66 may utilize an estimated pressure in combination with other respiratory parameters, such as resistance and compliance, to estimate flow.

The sensor estimator 66 utilizes the ongoing ventilator measurements taken by the ventilator 20 and the ventilator settings to simulate at least one parameter in the patient circuit 30. The sensor estimator 66 may be based on inputs received from patient 24, pneumatic system 22, sensors (e.g. a flow sensor 62 located outside of the breathing circuit 30), operator interface 52, and/or other components of the ventilator 20. In some embodiments, the sensor located outside of the breathing circuit 30 measures respiratory gases in a location outside of the breathing circuit but still in continuous flow of the respiratory gases from the breathing circuit, such as inside the pneumatic system 22. In other embodiments, the sensor is located outside of the breathing circuit and is not in continuous flow with the breathing circuit. The sensor estimator 66 can be stored in and utilized by the controller 50, by a computer system located in the pneumatic system 22, by a computer system located in the ventilator 20, or by an independent source that is operatively coupled with the pneumatic system 22 or ventilator 20.

The sensor estimator 66 may also interact with the signal measurement and acquisition systems, the controller 50 and the operator interface 52 to enable an operator to interact with the sensor estimator 66, the ventilator 20, and the display 59. Further, this coupling allows the controller 50 to receive and display the estimated patient sensor readings produced by the sensor estimator 66. This computer system may include memory, one or more processors, storage, and/or other components of the type commonly found in command and control computing devices. Furthermore, the sensor estimator 66 may be integrated into the ventilator 20 as shown, or may be a completely independent component residing on an external device such as another computing system).

As discussed above, flow sensors located in the patient circuit have hardware costs and operational issues. For instance, the sensors may be blocked from sending patient data during ventilation causing patient data gaps. However, the sensor estimator 66 (which may alternatively be referred to as a virtual proximal flow sensor, virtual sensor or virtual sensor module) estimates patient data, such as lung flow rate and airway pressure, in the patient circuit without the hardware costs or operational issues that are associated with a physical sensor. These estimates are saved, sent, and/or displayed by the ventilator and provide comparable information as obtained by a physical sensor. These estimates provide care-givers, patients, and the ventilators with continuously available information and allow for more informed patient treatment and diagnoses. In some embodiments, the sensor estimator 66 estimates the lung flow and/or airway pressure in the breathing circuit by utilizing at least one of ventilator settings, internal measurements, available hardware characteristics, and patient's respiratory mechanics parameters extracted from ventilatory data versus time in a fitting curve.

In other embodiments, a sensor estimator 66 utilizes a sensor model (or a bank of multiple models) that is designed and trained (values assigned to model parameters) to represent dynamics of the patient-ventilator system relevant to estimation of parameters of interest (e.g., flow, pressure). Further, in yet other embodiments, the sensor estimator 66 uses as inputs parameters based on the one or more fit parameters and at least one of the ventilator settings, the internal measurements, the available hardware characteristics, and the patient characteristics to provide sensor estimates of parameters in the breathing circuit 30.

In one embodiment, the sensor estimator 66 estimates the proximal flow and/or pressure at patient circuit wye 36 by utilizing the following model equations:

P_{y}(t)=Pₑₓₕ(t)+Q_{c}(t)*(K₁+K₂*Q_{c}(t)); and

Q_{c}(t)=Qₑₓₕ(t)+C_{ef}*Pₑ(t).

Wherein:

P_{y}=pressure at patient circuit wye extracted from ventilator data and circuit characteristics obtained through the ventilator calibration Self-Test process;

Q_{c}=flow rate in the exhalation limb, which is derived or calculated utilizing the above equation;

C_{ef}=compliance of exhalation filter and is a determined constant;

K₁, K₂=parameters of exhalation circuit limb resistance and are modeling parameters for the flow going through the circuit;

Pₑₓₕ=pressure at the exhalation port extracted from ventilator data;

Qₑₓₕ=flow at exhalation port extracted from ventilator data;

t=a continuous variable and stands for time in seconds as it elapses;

P_{y}(t)=the wye pressure estimate at time t; and

Pₑ=conditioned (filtered) time domain derivative of pressure (rate of change of pressure with time) measured at exhalation port, this may be calculated utilizing the following model equations in the frequency domain:

$${\dot{P}}_{e} ( s ) = \frac{s}{( s + {p}_{1} ) ( s + {p}_{2} ) ( β s + 1 )} {P}_{e} ( s ) ;$$

Q_{y}(s)=T₁(s)*Qᵥ(s)+T₂(s)*P_{y}(s)+E_{Qy}(s);

Pₑ=pressure at the exhalation port extracted from the ventilator;

Pₑ(s), Q_{y}(s), Qᵥ(s), Q_{del}(s), and E_{Qy}(s) are the Laplace transforms for the following:

Q_{y}(t)=estimated proximal flow at the patient circuit wye;

Qᵥ(t)=Q_{del}(t)−Qₑₓₕ(t);

Q_{del}(t)=total flow delivered by the ventilator;

E_{Qy}(t)=approximation residual or estimation error;

T₁(s)Qᵥ(s)=the Laplace transform of the contribution of the ventilator flow rate to the patient flow rate;

T₂(s)*P_{y}(s)=the Laplace transform of the contribution of pressure at patient circuit wye to patient flow rate;

$${T}_{1} ( s ) = d \frac{s + {z}_{1}}{( s + {p}_{3} ) ( s + {p}_{4} )} ;$$

and

$${T}_{2} ( s ) = - m * {T}_{1} ( s ) * \frac{s}{( s + {p}_{5} ) ( s + {p}_{6} )} .$$

s=Laplace variable;

z, p₁, p₂, p₃, p₄, p₅, and p₆=model parameters representing system dynamics

β=filtering parameter; and

d and m=modeling parameters.

Pₑ is used in the calculation of Q_{c} and P_{y} for Q_{y} estimation. The model parameters are dynamically updated based on ventilator settings, internal measurements (pressure, flow, etc.), available hardware characteristics, and estimated parameters of patient's respiratory mechanics extracted from ventilatory data. Additionally, one or more of these parameters may assume different values depending on the breath phase (inhalation or exhalation).

The models described above are but examples of how an estimate may be obtained based on the current settings and readings of the ventilator by the sensor estimator 66. Alternative models and model parameters and more involved modeling strategies (building a bank of models to serve different ventilator settings and/or patient conditions) may also be utilized by the sensor estimator 66. Furthermore, other wave-shaping modeling approaches and waveform quantifications and modeling techniques may be utilized by the sensor estimator 66 for hardware and/or respiratory parameter characterization. Parameters of such models may be dynamically updated and optimized during ventilation by the sensor estimator 66. Further, the sensor estimator 66 may utilize any of the models described in U.S. patent application Ser. No. 12/713,483, filed on Feb. 26, 2010, which is hereby incorporated by reference in its entirety.

The ventilator 20 includes a capnometer 48. As shown in FIG. 1, the capnometer 48 may be a separate component from ventilator 20. In other embodiments, the capnometer 48 may be an integral part of ventilator 20. Capnometer 48 is in data communication with ventilator 20. This communication allows the ventilator 20 and capnometer 48 to send data, instructions, and/or commands to each other. Capnometer 48 is in communication with processor 56 of ventilator 20.

Capnometer 48 monitors the concentrations of carbon dioxide in the respiratory gas with a carbon dioxide sensor at a location in the ventilator breathing circuit 30 (not shown). The carbon dioxide sensor may be located at the wye 36, near the patient interface, or at the same location being utilized to estimate a flow rate. The carbon dioxide sensor allows the capnometer 48 to monitor the concentrations of CO₂ in the gas transiting its sensor. Using a measured CO₂ in conjunction with an estimated flow calculated by the sensor estimator 66, the ventilator 20 can calculate volumetric carbon dioxide (VCO₂). In some embodiments, the capnometer 48 calculates the VCO₂ per breath. In other embodiments, VCO₂ is calculated by the ventilator 20, processor 56, controller 50, synchronization module 64, flow estimator 66, and/or pneumatic system 22. In further embodiments, information from the ventilator, such as inspiratory time, expiratory time, and/or breath period are utilized to identify integration limits for the calculation of VCO₂.

In addition to the measured CO₂ and estimated flow, the capnometer 48 or any other suitable ventilator component for calculating VCO₂ utilizes an algorithm to calculate the VCO₂ per breath. In some embodiments, the algorithm utilized to calculate VCO₂ per breath is listed below:

$${V CO}_{2} = {\sum }_{breath} {F}_{e} {CO}_{2} ( t ) * {\dot{V}}_{airway} ( t ) * Δ t$$

In other embodiments, capnometer 48 generates a capnogram with the measured CO₂. In further embodiments, the display 59 or any other suitable ventilator component displays the calculated volumetric CO₂, measured CO₂, the estimated flow measurement, the estimated pressure measurement, and/or the generated capnogram.

In some embodiments, ventilator 20 further includes a synchronization module 64 (the “Sync. Module” in FIG. 1). The location of the CO₂ sensor is not close to the location of the at least one sensor for measuring at least one respiratory parameter to determine the estimated flow, since the sensor is located outside of the breathing circuit. Accordingly, the CO₂ and the estimated flow rate may not be completely synchronized due to various factors, such as location, calculation delays, and transmission delays. Further, because a flow rate and a CO₂ concentration are multiplied by each other, any slight dyssynchrony may lead to significant errors in the VCO₂ calculation. Accordingly, the synchronization module 64 synchronizes a measurement taken by the capnometer with the estimated flow rate calculated by the sensor estimator 66 for a given sampling period to calculate a volumetric CO₂ per patient breath. Therefore, the synchronization module 64 eliminates or reduces errors caused by unsynchronized measurements to increase the accuracy of the volumetric CO₂ calculation.

The sampling period is determined by the timing of a common event and may include measurements taken or recorded at different times or measurements taken or recorded at the same time. Accordingly, the sampling period is a period or range of time that includes the time of the common event. In some embodiments, the common event is a start of inspiration, a start of exhalation, and/or a transition point between inspiration and exhalation. In some embodiments, the synchronization module 64 aligns at least one CO₂ measurement and at least one estimation of a flow rate based at least on the timing of the common event.

In some embodiments, if the common event is the start of inspiration, the synchronization module 64 determines the CO₂ measurement in the breathing circuit 30 at the start of inspiration and determines what the estimated flow rate was at the start of inspiration. While these two measurements may have been recorded by sensors at similar times, due to their different locations and different transmission and/or calculation delays, they may not have been recorded at the same time. Accordingly, the synchronization module 64 accounts for these delays to make sure the CO₂ measurement and the estimated flow rate were both taken at the time of the common event to align the measurements with the common event.

In other embodiments, the common event is utilized by the synchronization module 64 to determine a delay between the measurement of CO₂ and the estimated flow rate. The synchronization module 64 may account for the delay by aligning the measurement of CO₂ and the estimated flow.

For example, in other embodiments, the synchronization module 64 compares an estimated signal, such as a specific characteristic of the estimated respiratory flow signal, with a capnogram signal, which can both be recorded as waveforms. Next, in this embodiment, the synchronization module 64 picks a common point on the two different wave signals, such as the transition point between inspiration and expiration. While these signals are being recorded by sensors at similar times, due to their different locations and different transmission and/or calculation delays, they may not have the same time scale. Accordingly, the synchronization module 64 aligns the estimated flow signal with the capnogram based on the common wave point to account for these delays. This alignment may include delaying one wave signal until the common point of the capnogram aligns with the common point on the estimated respiratory flow rate wave signal.

In some embodiments, the synchronization module 64 utilizes the timing of the common event and other ventilator information to align the CO₂ measurement and the estimated flow rate, such as inspiratory status, expiratory status, response time of ventilator delivery valves, response time of ventilator exhalation valves, compliance of the breathing circuit, and/or estimates of anatomic dead-space. This list is exemplary only and is not limiting. Further, all of these embodiments are merely examples of how the ventilator 20 may synchronize a CO₂ measurement with an estimated flow rate. Other systems and methods for synchronizing a CO₂ measurement with an estimated flow rate may be utilized by the present disclosure.

In some embodiments, the ventilator 20 further includes a gas sensor other than a CO₂ sensor, such as an oxygen sensor, at a location in the breathing circuit 30. The gas sensor may be located at the wye 36, near the patient interface, or at the same location as the CO₂ sensor. The gas sensor monitors the concentration of the gas in the respiration gas in the breathing circuit 30 to determine various respiratory statuses, such as the start of exhalation, an inspiration/expiration signal, and/or the start of inhalation. In other embodiments, the synchronization module 64 utilizes the respiratory status based on the gas sensor measurements as the common event to synchronize the estimated flow rate with a CO₂ measurement to calculate the volumetric CO₂ per patient breath for the sampling period. A volumetric CO₂ calculated based on a CO₂ measurement synchronized with an estimated flow rate utilizing a respiratory status determined with a gas sensor may be more accurate than determining the respiratory status based merely on a CO₂ measurement and/or a flow rate measurement.

In further embodiments, when a volumetric CO₂ is calculated based on the use of the synchronization module 64, the sensor estimator 66, the controller 50, the processor 56, and/or the pneumatic system 22, the ventilator 20 may adjust the estimated lung flow based on this calculated volumetric CO₂ per patient breath. The use of this calculated VCO₂ with lung flow estimates may improve the accuracy of the lung flow estimates.

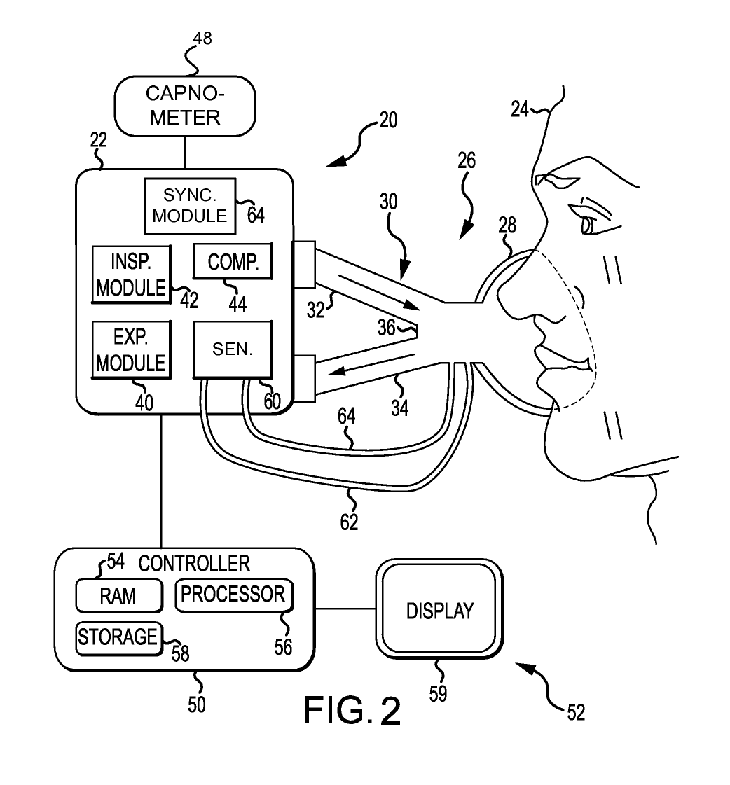

FIG. 2 illustrates an embodiment of ventilator 20 similar to FIG. 1, except that this ventilator 20 requires a synchronization module 64 and does not include a sensor estimator 66. Instead the ventilator 20 as illustrated in FIG. 2 includes a sensor 60 (the “SEN.” in FIG. 2) in the breathing circuit 30, such as a flow and/or pressure sensor. In some embodiments, the pressure sensor measurements in combination with other measured respiratory parameters, such as resistance and compliance, are utilized to calculate lung flow. The at least one sensor 60 monitors a respiratory parameter, such as flow rate and/or the airway pressure, at a location in the breathing circuit 30 in order to calculate the flow rate. In some embodiments, the location is at the wye 36 or near the patient interface 28.

While the location of the CO₂ sensor may be close to or at the same location as the sensor 60 in the breathing circuit 30, their measurements may not be completely synchronized due to various factors, such as calculation delays, and transmission delays. Further, because the flow rate measurement and CO₂ rate are multiplied by each other to calculate VCO₂, any changes caused by dyssynchrony will lead to errors in accuracy of a VCO₂ calculation. Accordingly, the synchronization module 64 in FIG. 2, synchronizes a measurement taken by the capnometer with the measured flow rate in the breathing circuit 30 (instead of an estimated flow rate as discussed above in FIG. 1) from a same sampling period to calculate volumetric CO₂ per patient breath. Therefore, the synchronization module 64 eliminates or minimizes the impact of changes caused by unsynchronized measurements to increase the accuracy of the volumetric CO₂ calculation.

The sampling period, as discussed above, is determined by the timing of a common event and may include measurements taken or recorded at different times or measurements taken or recorded at the same time. Accordingly, the sampling period is a period or range of time that includes the time of the common event. In some embodiments, the common event is a start of inspiration, a start of exhalation, and/or a transition point between inspiration and exhalation. In some embodiments, the synchronization module 64 aligns the CO₂ measurement and the measured flow based at least on the timing of the common event.

In some embodiments, if the common event is the start of inspiration, the synchronization module 64 determines the CO₂ measurement in the breathing circuit 30 at the start of inspiration and determines what the flow rate measurement was at the start of inspiration. While these two measurements may have been recorded by sensors at similar times and at similar locations, due to their different transmission and/or calculation delays, they may not have been recorded at the same time. Accordingly, the synchronization module 64 accounts for these delays to make sure the CO₂ measurement and the flow rate measurement were both taken at the time of the common event to align the measurements with the common event.

In other embodiments, the common event is utilized by the synchronization module 64 to determine a delay between the measurement of CO₂ and the flow rate measurement. The synchronization module 64 may account for the delay to align the measurement of CO₂ and the flow measurement.

For example, in other embodiments, the synchronization module 64 compares a respiratory flow wave signal, such as a flow wave signal, with a capnogram, which can both be recorded as waveforms. Next, in this embodiment, the synchronization module 64 picks a common point on the two different wave signals, such as the transition point between inspiration and expiration. While these signals are being recorded by sensors at similar times and locations, due to different transmission and/or calculation delays, they may not have the same time scale. Accordingly, the synchronization module 64 aligns the flow signal with the capnogram based on the common wave point to account for these delays. This alignment may include delaying one wave signal until the common point of the capnogram aligns with the common point on the flow rate wave signal.

In some embodiments, as discussed above, the synchronization module 64 utilizes the timing of the common event and other ventilator information, such as inspiratory status, expiratory status, response time of ventilator delivery valves, response time of ventilator exhalation valves, compliance of the breathing circuit, and/or estimates of anatomic dead-space, to align the CO₂ measurement and the measured flow. Further all of these embodiments are merely examples of how the ventilator 20 may synchronize a CO₂ measurement with a flow rate measurement. Other systems and methods for synchronizing a CO₂ measurement with a flow rate measurement may be utilized by the present disclosure.

In some embodiments, the ventilator 20 further includes a gas sensor other than a CO₂ sensor, such as an oxygen sensor, at a location in the breathing circuit 30. The gas sensor may be located at the wye 36, near the patient interface, or at the same location as the CO₂ sensor. The gas sensor monitors the concentration of the gas in the respiration gas in the breathing circuit 30 to determine various respiratory statuses, such as the start of exhalation and the start of inhalation. In other embodiments, the synchronization module 64 utilizes the respiratory status based on the gas sensor measurements as the common event to synchronize the flow rate measurement with a CO₂ measurement to calculate the volumetric CO₂ per patient breath for the sampling period. A volumetric CO₂ calculated based on a CO₂ measurement synchronized with a flow rate measurement utilizing a respiratory status determined with a gas sensor may be more accurate than determining the respiratory status based merely on a CO₂ measurement and/or a flow rate measurement.

In some embodiments, both ventilators 20 illustrated in FIGS. 1 and 2 and described above provide for a volumetric CO₂ per patient breath with an accuracy of at least 15% within the actual amount of CO₂ being produced per patient breath. In other embodiments, both ventilators 20 illustrated in FIGS. 1 and 2 and described above provide for a volumetric CO₂ per patient breath with an accuracy of at least 10% within the actual amount of CO₂ being produced per patient breath. In further embodiments, both ventilators 20 illustrated in FIGS. 1 and 2 and described above provide for a volumetric CO₂ per patient breath with an accuracy of at least 5% within the actual amount of CO₂ being produced per patient breath.

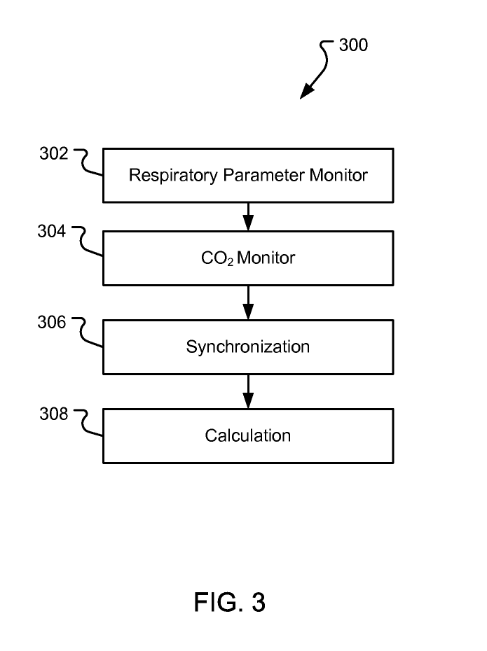

FIG. 3 illustrates an embodiment of a method for monitoring volumetric CO₂ during ventilation of a patient by a medical ventilator. As illustrated, the monitoring method 300 includes a respiratory parameter monitor operation 302. The ventilator during the respiratory parameter monitor operation 302, monitors a respiratory parameter, such as lung flow, within a breathing circuit with at least one sensor at a location within the breathing circuit. The sensor may be any suitable sensor for measuring the respiratory parameter within the breathing circuit of the ventilator, such as flow and/or pressure sensor. The pressure sensor in combination with other measured respiratory parameters, such as resistance and compliance, may be utilized to calculate lung flow. In some embodiments, the location of the sensor is at the wye of the breathing circuit. In other embodiments, the location of the sensor is near a patient interface in the breathing circuit.

Further, method 300 includes a CO₂ monitor operation 304. During the CO₂ monitor operation 304, the ventilator monitors CO₂ concentrations with a capnometer at a location in the breathing circuit. The capnometer may include any suitable sensor for measuring the amount of CO₂ within the breathing circuit of the ventilator. In some embodiments, location of the capnometer sensor is at the wye of the breathing circuit. In other embodiments, the location of the capnometer sensor is near a patient interface in the breathing circuit. In some embodiments, the location of the capnometer sensor is near or at the same location as the respiratory parameter sensor in the breathing circuit.

Method 300 includes a synchronization operation 306. The ventilator during the synchronization operation 306, synchronizes a CO₂ measurement taken by the capnometer with a flow rate measurement taken by the sensor from a same sampling period. The sampling period, as discussed above, is determined by the timing of a common event and may include measurements taken or recorded at different times or measurements taken or recorded at the same time. Accordingly, the sampling period is a period or range of time that includes the time of the common event.

In some embodiments, during the synchronization operation 306, the ventilator may perform a selection operation and an alignment operation. The ventilator during the selection operation selects a common event. In some embodiments, the common event is a start of inspiration, a start of exhalation, and/or a transition point between inspiration and exhalation.

The ventilator during the alignment operation synchronizes or aligns the CO₂ measurement and the flow rate measurement based at least on the timing of the selected common event. For example, the ventilator during the alignment operation may utilize the common event to determine a delay between the measurement of CO₂ and the flow rate measurement. In this embodiment, the ventilator during the alignment operation accounts for the delay to align or synchronize the measurement of CO₂ and the flow rate measurement based on the common event.

For example, in some embodiments, the ventilator during the selection operation selects the start of inspiration as the common event. In this embodiment, the ventilator during the alignment operation determines the CO₂ measurement in the breathing circuit measured at the start of inspiration and determines the flow rate measurement measured at the start of inspiration. While these two measurements may have been recorded by sensors at similar times and at similar locations, due to their different transmission and/or calculation delays, they may not have been recorded at the same time. Accordingly, the ventilator during the alignment operation accounts for these delays to make sure the CO₂ measurement and the flow rate measurement were both taken at the time of the common event to align the measurements.

In other embodiments, the ventilator during the selection operation selects a common point on a respiratory flow wave signal and on a capnogram, such as the transition point between inspiration and expiration. The respiratory flow wave signal may be a flow waveform. In this embodiment, the ventilator during the alignment operation compares the respiratory flow wave signal with a capnogram, which can both be recorded as waveforms. While these signals are being recorded by sensors at similar times and locations, due to different transmission and/or calculation delays, they may not have the same time scale. Accordingly, the ventilator during the alignment operation aligns or synchronizes the respiratory flow wave signal with the capnogram based on the common wave point to account for these delays. This alignment may include delaying one wave signal until the common point of the capnogram aligns with the common point on the respiratory flow wave signal.

In some embodiments, the ventilator during the synchronization operation 306 aligns the CO₂ measurement and the flow rate measurement based on the timing of the common event and based on other ventilator information, such as inspiratory status, expiratory status, response time of ventilator delivery valves, response time of ventilator exhalation valves, compliance of the breathing circuit, and/or estimates of anatomic dead-space. This list is exemplary only and is not limiting. Further, all of these embodiments are merely examples of how the ventilator 20 may synchronize a CO₂ measurement with a flow rate measurement. Other systems and method for synchronizing a CO₂ measurement with a flow rate measurement may be utilized by the present disclosure.

Additionally, method 300 includes a calculation operation 308. During the calculation operation 308, the ventilator calculates the volumetric CO₂ passing through at one of the location of the CO₂ sensor and/or the respiratory parameter sensor for at least one breath based at least on an algorithm and the at least one CO₂ measurement synchronized with the at least one flow rate measurement. In some embodiments, the algorithm is the following:

$${V CO}_{2} = {\sum }_{breath} {F}_{e} {CO}_{2} ( t ) * {\dot{V}}_{airway} ( t ) * Δ t .$$

In further embodiments, the ventilator during calculation operation 308 utilizes ventilator information, such as inspiratory time, expiratory time, and/or breath period, to identify integration limits for the calculation of VCO₂.

As discussed above, because the flow rate measurement and CO₂ measurement are multiplied by each other, any slight changes caused by unsynchronized measurements will be exponentially magnified after the multiplying of these two measurements decreasing the accuracy of a VCO₂ calculation. Accordingly, the synchronization operation 306 eliminates or reduces slight changes caused by unsynchronized measurements to increase the accuracy of the volumetric CO₂ calculation. In some embodiments, a ventilator performing the method 300 provides for a volumetric CO₂ per patient breath with an accuracy of at least 15% within the actual amount of CO₂ being produced per patient breath. In other embodiments, a ventilator performing method 300 provides for a volumetric CO₂ per patient breath with an accuracy of at least 10% within the actual amount of CO₂ being produced per patient breath. In further embodiments, a ventilator performing method 300 provides for a volumetric CO₂ per patient breath with an accuracy of at least 5% within the actual amount of CO₂ being produced per patient breath.

In some embodiments, method 300 includes a gas monitor operation, which may be an independent operation or included with the CO₂ monitor operation 304. The ventilator, during the gas monitor operation, monitors an amount of gas other than CO₂, such as oxygen, exhaled by the patient with a gas sensor at a location in the breathing circuit. The location of the gas sensor may be at the wye, near the patient interface, and/or at the same location as the CO₂ sensor. During these embodiments, the ventilator determines a respiratory status, such as the start of inspiration or the start of exhalation, based on the gas concentration measurements. The ventilator during the synchronization operation 306 may utilize the respiratory status information calculated based on the gas sensor measurements alone and/or in addition to other respiratory parameters to determine a common event for synchronizing the CO₂ and flow rate measurements.

In further embodiments, method 300 includes a display operation. The ventilator during the display operation displays the calculated volumetric CO₂. In some embodiments, the ventilator during the display operation may further display measured CO₂, the flow measurement, the pressure measurement, the common event, the delay, and/or a generated capnogram.

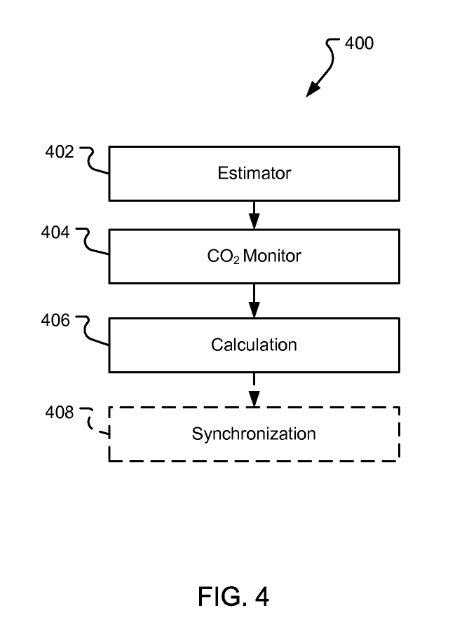

FIG. 4 illustrates an embodiment of a method for monitoring volumetric CO₂ during ventilation of a patient being ventilated by a medical ventilator, 400. As illustrated, method 400 includes an estimator operation 402. The ventilator during the estimator operation 402 estimates at least one flow rate at a location in a breathing circuit by monitoring at least one respiratory parameter with at least one sensor located outside of the breathing circuit. In some embodiments, the respiratory parameter is lung flow and/or pressure. The monitored pressure in combination with other ventilatory parameters, such as resistance and compliance, may be utilized to calculate the estimated flow rate. In some embodiments, the location in the breathing circuit is near the patient airway in the breathing circuit. In other embodiments, the location in the breathing circuit is at the wye of a breathing circuit. The sensor may be any suitable sensor for measuring the flow rate within the ventilator and separate from the breathing circuit. In some embodiments, the sensor is at least one of a flow sensor and pressure sensor.

In some embodiments, the ventilator during the estimator operation 402 estimates the flow rate by utilizing current and/or past ventilator settings, internal measurements, available hardware characteristics, and patient's respiratory mechanics parameters extracted from ventilator data to generate the estimates. In other embodiments, the ventilator during the estimator operation 402 estimates the flow rate by utilizing a model for the system. The model may be any suitable model as long as it can provide a reasonably accurate prediction of the flow rate in the breathing circuit based on past patient circuit wye estimates and current and/or past ventilator sensor readings. In further embodiments, the model equations (in time and frequency domains) for the modeling process are:

P_{y}(t)=Pₑₓₕ(t)+Q_{c}(t)*(K₁+K₂*Q_{c}(t));

Q_{c}(t)=Qₑₓₕ(t)+C_{ef}*Pₑ(t);

$${\dot{P}}_{e} ( s ) = \frac{s}{( s + {p}_{1} ) ( s + {p}_{2} ) ( β s + 1 )} {P}_{e} ( s ) ;$$

Q_{y}(s)=T₁(s)*Qᵥ(s)+T₂(s)*P_{y}(s)+E_{Qy}(s);

$${T}_{1} ( s ) = d \frac{s + {z}_{1}}{( s + {p}_{3} ) ( s + {p}_{4} )} ;$$

and

$${T}_{2} ( s ) = - m * {T}_{1} ( s ) * \frac{s}{( s + {p}_{5} ) ( s + {p}_{6} )} .$$

Further, the model may be any model described in U.S. patent application Ser. No. 12/713,483, filed on Feb. 26, 2010, which is hereby incorporated by reference in its entirety.

Further, method 400 includes a CO₂ monitor operation 404. During the CO₂ monitor operation 404, the ventilator monitors CO₂ concentrations with a capnometer at a location in the breathing circuit. The capnometer may include any suitable sensor for measuring the amount of CO₂ within the breathing circuit of the ventilator. In some embodiments, the location of the capnometer sensor is at the wye of the breathing circuit. In other embodiments, the location of the capnometer sensor is near a patient interface in the breathing circuit. In some embodiments, the location of the capnometer sensor is near or at the same location for the estimated flow rate in the breathing circuit. In any case, the capnometer sensor may or may not be located at the same location as that for which the estimator operation 402 is estimating the flow rate.

Additionally, method 400 includes a calculation operation 406. The ventilator during the calculation operation 406, calculates a volumetric CO₂ passing through at least one of the locations for at least one breath based at least on an algorithm, the monitored CO₂ concentrations taken by the capnometer, and the at least one estimated flow rate. In some embodiments, the algorithm is the following:

$${V CO}_{2} = {\sum }_{breath} {F}_{e} {CO}_{2} ( t ) * {\dot{V}}_{airway} ( t ) * Δ t .$$

In further embodiments, the ventilator during calculation operation 406 utilizes ventilator information, such as inspiratory time, expiratory time, and/or breath period, to identify integration limits for the calculation of VCO₂.

In some embodiments, method 400 further includes a synchronization operation 408. The ventilator during the synchronization operation 408, synchronizes at least one CO₂ measurement taken by the capnometer with the at least one estimated flow rate from a same sampling period. The sampling period, as discussed above, is determined by the timing of a common event and may include measurements taken or recorded at different times or measurements taken or recorded at the same time. Accordingly, the sampling period is a period or range of time that includes the time of the common event.

In some embodiments, during the synchronization operation 408, the ventilator may perform a selection operation and an alignment operation. The ventilator during the selection operation selects a common event. In some embodiments, the common event is a start of inspiration, a start of exhalation, and/or a transition point between inspiration and exhalation.

The ventilator during the alignment operation synchronizes or aligns the CO₂ measurement and the estimated flow rate based at least on the timing of the selected common event. For example, the ventilator during the alignment operation may utilize the common event to determine a delay between the measurement of CO₂ and the estimated flow rate. In this embodiment, the ventilator during the alignment operation accounts for the delay to align or synchronize the measurement of CO₂ and the estimated flow rate based on the common event.

For example, in some embodiments, the ventilator during the selection operation selects the start of inspiration at the common event. In this embodiment, the ventilator during the alignment operation determines the CO₂ measurement in the breathing circuit measured at the start of inspiration and determines the estimated flow rate at the start of inspiration. While the CO₂ measurement and estimated flow rate may have been recorded by sensors at similar times, due to their different locations and different transmission and/or calculation delays, they may not have been recorded at the same time. Accordingly, the ventilator during the alignment operation accounts for these delays to make sure the CO₂ measurement and the flow rate were both taken at the time of the common event to align the measurements.

In other embodiments, the ventilator during the selection operation selects a common point on an estimated respiratory flow wave signal and on a capnogram, such as the beginning of exhalation. The estimated flow rate wave signal may be an estimated flow waveform. In this embodiment, the ventilator during the alignment operation compares the estimated respiratory flow wave signal with the capnogram, which can both be recorded as waveforms. While these signals may be recorded by sensors at similar times, due to their different locations and different transmission and/or calculation delays, they may not have the same time scale. Accordingly, the ventilator during the alignment operation aligns or synchronizes the estimated respiratory flow wave signal with the capnogram based on the common wave point to account for these delays. This alignment may include delaying one wave signal until the common point of the capnogram aligns with the common point on the estimated respiratory flow wave signal.

In some embodiments, the ventilator during the synchronization operation 408 aligns the CO₂ measurement and the estimated flow based on the timing of the common event and based on other ventilator information, such as inspiratory status, expiratory status, response time of ventilator delivery valves, response time of ventilator exhalation valves, compliance of the breathing circuit, and/or estimates of anatomic dead-space. This list is exemplary only and is not limiting. Further, all of these embodiments are merely examples of how the ventilator may synchronize a CO₂ measurement with an estimated flow rate. Other systems and method for synchronizing a CO₂ measurement with an estimated flow rate may be utilized by the present disclosure.

As discussed above, because the estimated flow rate and CO₂ are multiplied by each other, any slight changes caused by unsynchronized measurements will be exponentially magnified after the multiplying of these two measurements decreasing the accuracy of a VCO₂ calculation. Accordingly, the synchronization operation 408 eliminates or reduces slight changes caused by unsynchronized measurements to increase the accuracy of the volumetric CO₂ calculation.

In further embodiments, method 400 includes a display operation. The ventilator during the display operation displays the calculated volumetric CO₂. In some embodiments, the ventilator during the display operation further displays measured CO₂, the estimated flow, the estimated pressure, the common event, the delay, and/or a generated capnogram.

In some embodiments, a ventilator performing method 400 provides for a volumetric CO₂ per patient breath with an accuracy of at least 15% within the actual amount of CO₂ being produced per patient breath. In other embodiments, a ventilator performing method 400 provides for a volumetric CO₂ per patient breath with an accuracy of at least 10% within the actual amount of CO₂ being produced per patient breath. In further embodiments, a ventilator performing method 400 provides for a volumetric CO₂ per patient breath with an accuracy of at least 5% within the actual amount of CO₂ being produced per patient breath.

In other embodiments, method 400 further includes a gas monitor operation. The ventilator during the gas monitor operation, monitors an amount of a gas other than CO₂, such as oxygen, exhaled by the patient with a gas sensor at a location in the breathing circuit. The location of the gas sensor may be at the patient wye, near the patient interface, or near or at the same location as the CO₂ sensor. During these embodiments, the ventilator determines a respiratory status, such as the start of inspiration or the transition point between inhalation and exhalation, based on the gas concentration measurements. The ventilator during the synchronization operation 408 may utilize the respiratory status information calculated based on the gas sensor measurements alone or in addition to other respiratory parameters to determine a common event for synchronizing the CO₂ measurements with estimated flow rates.

In some embodiments, a microprocessor-based ventilator that accesses a computer-readable medium having computer-executable instructions for performing the method of monitoring volumetric CO₂ during ventilation of a patient being ventilated by a medical ventilator is disclosed. This method includes repeatedly performing the steps disclosed in methods 300 or 400 and as illustrated in FIGS. 3 and 4.

In some embodiments, the ventilator system includes: means for estimating at least one flow rate at a first location in a breathing circuit by monitoring at least one respiratory parameter with at least one sensor located outside of the breathing circuit; means for monitoring CO₂ concentrations with a capnometer at a second location in the breathing circuit; and means for calculating a volumetric CO₂ passing through at least one of the first and second locations for at least one breath based at least on an algorithm, the monitored CO₂ concentrations taken by the capnometer, and the at least one estimated flow rate.

In some embodiments, the ventilator system includes: means for monitoring flow rate with at least one sensor at a first location within a breathing circuit; means for monitoring CO₂ concentrations with a capnometer at a second location in the breathing circuit; means for synchronizing at least one CO₂ measurement taken by the capnometer with at least one flow rate measurement taken by the at least one sensor from a same sampling period; and means for calculating a volumetric CO₂ passing through at least one of the first and second location for at least one breath based at least on an algorithm and the at least one CO₂ measurement synchronized with the at least one flow rate measurement.

### Examples

Several experiments were run to determine benefits and accuracy of synchronizing a CO₂ measurement with an estimated flow rate calculated with a ventilator. The example below documents a representative example of these experiments and their results.

The experiments were conducted to determine the feasibility of measuring VCO₂ using the estimated lung flow from the 840 Ventilator sold by Covidien-Nellcor and Puritan Bennett, located at 6135 Gunbarrel Avenue, Boulder, Colo. 80301, and the CO₂ concentration from a Philips Capnostat 5 sensor, sold by Philips Healthcare located at 3000 Minuteman Road, Andover, Mass. 01810-1099.

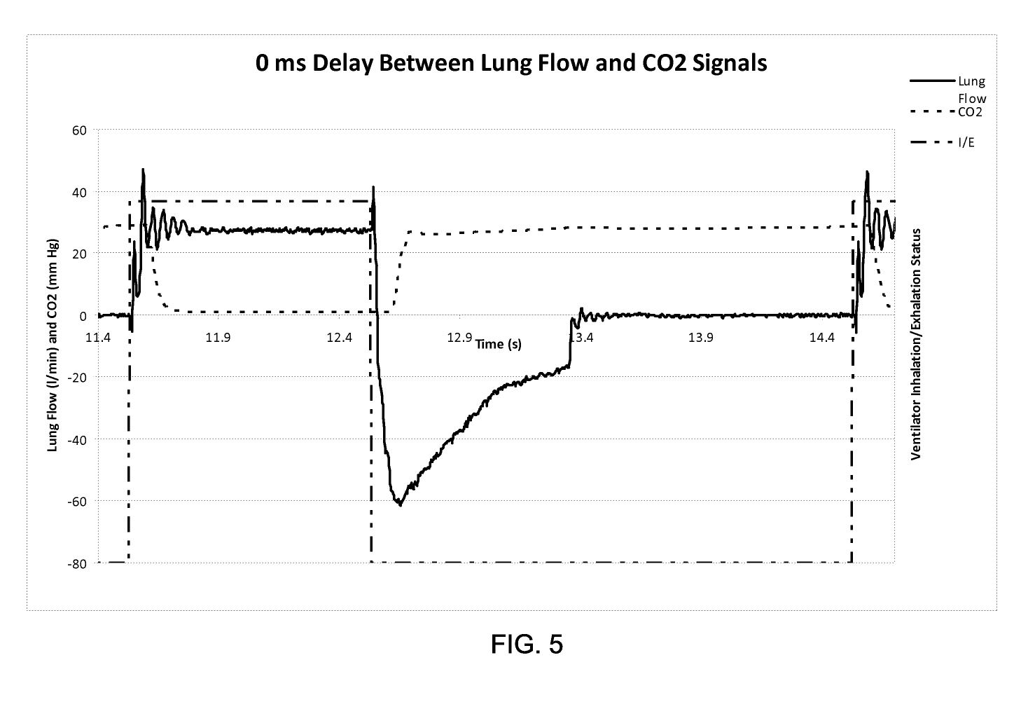

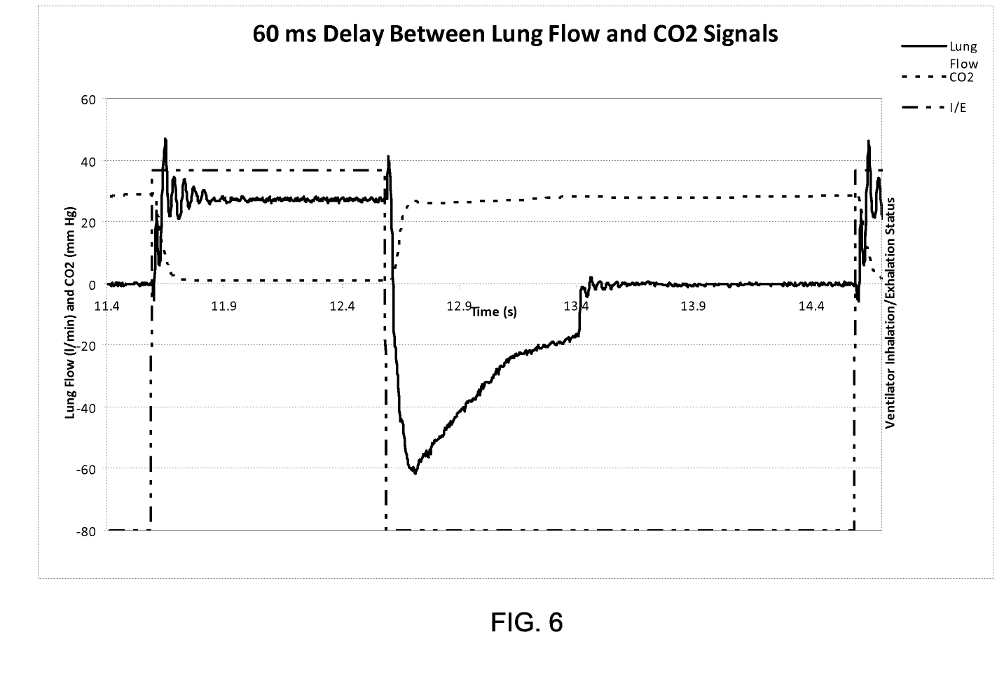

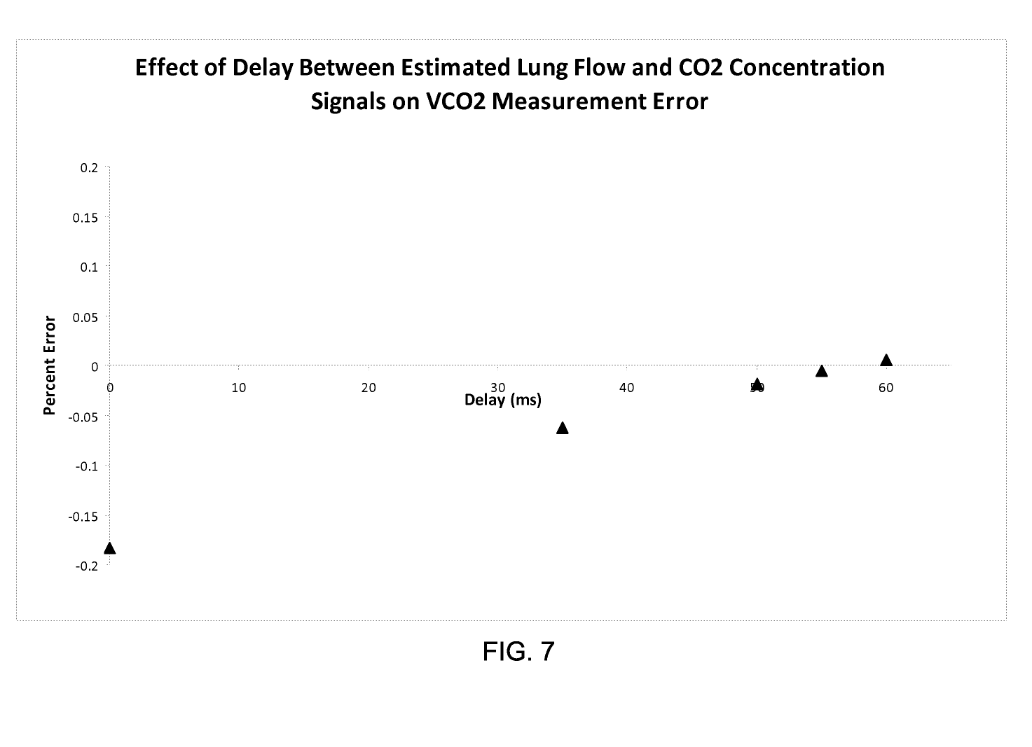

Using a test lung model, elimination of CO₂ was simulated by bleeding USP Grade carbon dioxide into the lung chamber. Signals for estimated lung flow, ventilator inhalation/exhalation status, and concentration of CO₂ were collected from the ventilator and CO₂ sensor. The volumetric CO₂ was then calculated for various delays between the two signals. FIG. 5 shows the signals collected for a single breath with data as collected (i.e., 0 ins delay). FIG. 6 shows the same signals when a delay of 60 ms is applied. FIG. 7 shows the effect of applying various signal delays on the error in the VCO₂ measurement. As shown in FIG. 7, the use of the estimated lung flow signal and the appropriate delay with the CO₂ signal results in a reduction in error from −18.3% to 0.6%.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the claims.

What is claimed is:

1. A method for monitoring volumetric CO₂ during ventilation of a patient being ventilated by a medical ventilator, the method comprising:
estimating at least one flow rate at a first location in a breathing circuit by monitoring at least one respiratory parameter with at least one sensor located outside of the breathing circuit;
monitoring CO₂ concentrations with a capnometer at a second location in the breathing circuit; and
calculating a volumetric CO₂ passing through at least one of the first and second locations for at least one breath based at least on an algorithm, the monitored CO₂ concentrations taken by the capnometer, and the at least one estimated flow rate.

2. The method of claim 1, wherein the at least one monitored respiratory parameter is at least one of flow and pressure.

3. The method of claim 1, wherein the first location and the second location are the same location.

4. The method of claim 1, wherein the algorithm is
$${V CO}_{2} = {\sum }_{breath} {F}_{e} {CO}_{2} ( t ) * {\dot{V}}_{airway} ( t ) * Δ t .$$

5. The method of claim 1, further comprising synchronizing at least one CO₂ measurement taken by the capnometer with the at least one estimated flow rate from a same sampling period.

6. The method of claim 5, further comprising monitoring an amount of oxygen exhaled by the patient with an oxygen sensor at a third location in the breathing circuit.

7. The method of claim 6, wherein the step of synchronizing is at least based on at least one oxygen measurement taken by the oxygen sensor.

8. The method of claim 5, wherein the step of calculating the volumetric CO₂ for each breath has an accuracy of at least 90%.

9. The method of claim 5, wherein the step of synchronizing comprises:
selecting a common event; and
aligning the at least one measurement of CO₂ and the at least one estimated flow rate based at least on timing of the common event.

10. The method of claim 9, wherein the step of aligning further comprises utilizing the common event is to determine a delay between the at least one CO₂ measurement and the at least one estimated parameter.

11. The method of claim 10, wherein the step of aligning further comprises accounting for the delay to synchronize the at least one CO₂ measurement with the at least one estimated parameter.

12. The method of claim 11, wherein the common event is at least one of a start of inspiration, a start of exhalation, and a transition point between inspiration and exhalation.

13. The method of claim 11, wherein the step of aligning is further based on at least one of inspiratory status, expiratory status, response time of ventilator delivery valves, response time of ventilator exhalation valves, compliance of the breathing circuit, and estimates of anatomic dead-space.

14. The method of claim 5, further comprising utilizing the calculated volumetric CO₂ to adjust the at least one estimated parameter.

15. A medical ventilator system, comprising:
a pneumatic gas delivery system, the pneumatic gas delivery system adapted to control a flow of gas from a gas supply to a patient via a breathing circuit;
a sensor estimator, the sensor estimator estimates at least one flow rate at a first location in the breathing circuit based at least on measurements taken by at least one sensor located outside of the breathing circuit;
a capnometer, the capnometer monitors an amount of carbon dioxide in respiration gas at a second location in the breathing circuit; and
a processor in communication with the pneumatic gas delivery system, the sensor estimator, and the capnometer, the processor is configured to calculate a volumetric CO₂ passing through at least one of the first and second locations for at least one breath based at least on an algorithm, the monitored CO₂ concentrations taken by the capnometer, and the at least one estimated flow rate.

16. The medical ventilator system of claim 15, wherein the at least one sensor is at least one of a flow sensor and a pressure sensor.

17. The medical ventilator system of claim 15, wherein the first location and the second location are the same location.

18. The medical ventilator system of claim 15, further comprising a synchronization module, the synchronization module synchronizes at least one CO₂ measurement taken by the capnometer with the at least one estimated parameter from a same sampling period.

19. The medical ventilator system of claim 18, further comprising an oxygen sensor, the oxygen sensor monitors the amount of oxygen in the respiration gas at a third location in the breathing circuit.

20. The medical ventilator system of claim 19, wherein the synchronization module further synchronizes the at least one CO₂ measurement with the at least one estimated flow rate from the same sampling period based at least on at least one oxygen measurement taken by the oxygen sensor.

21. The medical ventilator system of claim 19, wherein the sampling period is determined by a timing of a common event.

22. The medical ventilator system of claim 21, wherein the common event is at least one of a start of inspiration, a start of exhalation, and a transition point between inspiration and exhalation.

23. A computer-readable medium having computer-executable instructions for monitoring volumetric CO₂ during ventilation of a patient being ventilated by a medical ventilator, the method comprising:
repeatedly estimating at least one flow rate at a first location in a breathing circuit by monitoring at least one respiratory parameter with at least one sensor located outside of the breathing circuit;
repeatedly monitoring CO₂ concentrations with a capnometer at a second location in the breathing circuit; and
repeatedly calculating a volumetric CO₂ passing through at least one of the first and second locations for at least one breath based at least on an algorithm, the monitored CO₂ concentrations taken by the capnometer, and the at least one estimated flow rate.

24. A medical ventilator system, comprising:
means for estimating at least one flow rate at a first location in a breathing circuit by monitoring at least one respiratory parameter with at least one sensor located outside of the breathing circuit;
means for monitoring CO₂ concentrations with a capnometer at a second location in the breathing circuit; and
means for calculating a volumetric CO₂ passing through at least one of the first and second locations for at least one breath based at least on an algorithm, the monitored CO₂ concentrations taken by the capnometer, and the at least one estimated flow rate.
